(19) United States
(12) Patent Application Publication
(10) Pub. No.: US 2026/0186528 A1
(43) Pub. Date: Jul. 2, 2026

(54) SANITIZING CASE AND METHOD FOR A PHONE OR OTHER MOBILE DEVICE

(71) Applicant: Germ Dome Industries LLC, Orchard Lake, MI (US)

(72) Inventor: Dean Schumacher, Orchard Lake, MI (US)

(21) Appl. No.: 18/770,368

(22) PCT Filed: Jan. 11, 2023
(86) PCT No.: PCT/US2023/010600

§ 371 Date: Jul. 11, 2024

Related U.S. Application Data
(60) Provisional application No. 63/298,467, filed on Jan. 11, 2022.

(51) Int. Cl. G06F 1/16; A61L 2/104; A61L 2/26; H04M 1/02

(52) U.S. Cl. CPC G06F 1/1629; A61L 2/104; A61L 2/26; G06F 1/1656; A61L 2202/11; A61L 2202/16; H04M 1/0203

(57) ABSTRACT

A mobile electronic device case includes a body, an arm movably coupled to the body, and a germicidal agent source disposed on the arm. The body defines an interior configured to secure a mobile electronic device. The arm is movable relative to the mobile electronic device for applying the germicidal agent to the mobile electronic device when the mobile electronic device is secured in the body.

### BACKGROUND

### a. Technical Field

The present disclosure relates to a system and apparatus for a case to secure a phone or similar mobile device. More specifically, this disclosure relates to a system and apparatus for sanitizing the surfaces of a cell phone, tablet, portable music device, portable gaming system, or another mobile device.

### SUMMARY

A mobile electronic device case may include a body, an arm movably coupled to the body, and a germicidal agent source disposed on the arm. The body defines an interior that is configured to secure a mobile electronic device. The arm is movable relative to the mobile electronic device for applying the germicidal agent to the mobile electronic device when the mobile electronic device is secured in the body.

Another mobile electronic device case may include a body that defines an interior and a track, an arm movably coupled to the body, and a germicidal agent source disposed on the arm. The interior is configured to secure a mobile electronic device. The arm is slidably and rotatably coupled to the track, and movable relative to the mobile electronic device for applying the germicidal agent to each exposed surface of the case and of the mobile electronic device when the mobile electronic device is secured in the body.

A method for sanitizing a mobile electronic device includes providing a case for the mobile electronic device. The case includes a body that defines an interior and a track, an arm movably coupled to the body, and a germicidal agent source disposed on the arm. The interior is configured to secure a mobile electronic device. The method further includes activating the germicidal agent source and moving the arm along the track to sanitize one or more surfaces of the case and of the mobile electronic device

### BRIEF DESCRIPTION OF THE DRAWINGS

FIG. 1 is an isometric view of a first configuration of an example sanitizing case for a mobile device;

FIG. 2 is an isometric view of a second configuration of the example sanitizing case;

FIG. 3 is an isometric view of a third configuration of the example sanitizing case;

FIG. 4 is an isometric view of a fourth configuration of the example sanitizing case;

FIG. 5 is an isometric view of a fifth configuration of the example sanitizing case;

FIG. 6 is an isometric view of a sixth configuration of the example sanitizing case;

FIG. 7 is an isometric view of a seventh configuration of the example sanitizing case;

FIG. 8 is a side view of the third configuration of the example sanitizing case;

FIG. 9 is a isometric view of an arm of the example sanitizing case;

FIG. 10 is a side view of the third configuration of the example sanitizing case;

FIGS. 11-19 are further views of an example sanitizing case for a mobile device;

FIG. 20 includes various views of an example sanitizing case for a mobile device; and

FIGS. 21-26 are various views of an example sanitizing case for a mobile device.

### DETAILED DESCRIPTION

Cell phones and similar mobile devices can be involved in the spread of communicable germs. Phones are continuously touched, particularly when in use, and can serve to attract and retain germs as a result of those touches. Phones are often passed around between users, and contact from each user can multiply the number of germs on the phone. When not in use, phones are often left on tables, desks, seats, or other various surfaces, which themselves have germs that can be passed to the phone.

The effect of germs spread through phones can be significantly reduced by sterilizing or sanitizing the surfaces of the phone periodically, such as after each use of the phone. There is therefore a need for a system and apparatus to sanitize phone surfaces to eliminate one or more of the problems described above.

Certain sanitizing and germicidal agents may be used to sanitize objects and surfaces, such as cell phones and other mobile electronic devices. One type of sanitizing agent is electromagnetic energy of a particular frequency in the ultraviolet (UV) light spectrum. Ultraviolet light with a wavelength in the range of about 100 nanometers to about 280 nanometers (referred to herein as germicidal UV) is known to be deadly to bacteria and other micro-organisms, killing the micro-organisms, rendering them harmless, or eliminating their ability to reproduce. Germicidal UV with a wavelength of about 254 nanometers (a frequency of about 1.18 petahertz (PHz)) is known to be particularly effective. In one embodiment of the present invention, germicidal UV may be used as an effective agent for sterilizing or sanitizing the surfaces of phones, and other mobile devices that may be points of contact for multiple users, referred to herein as “points of contact.” Similarly, as used herein, a “point-of-contact surface” refers to any surface of a point of contact (e.g., a phone screen, a back of a phone, buttons on a gaming device, etc.). A system and apparatus for sanitizing such point-of-contact surfaces may involve germicidal UV or other sanitizing agents.

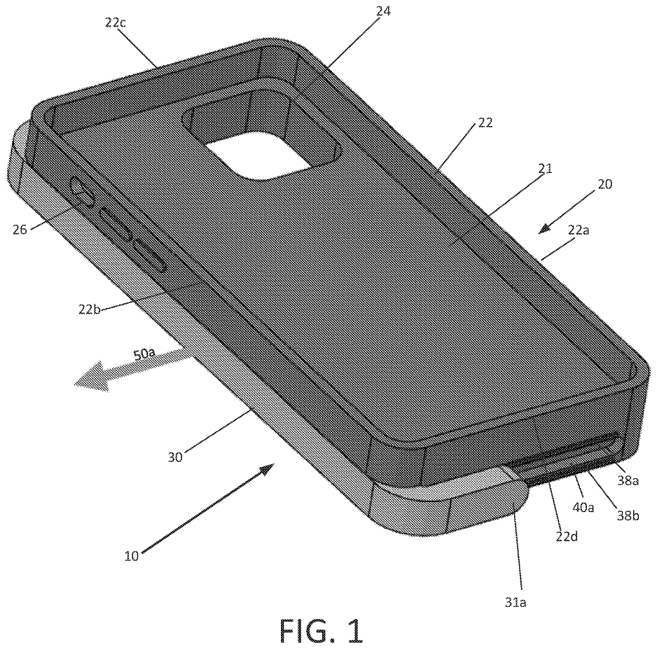

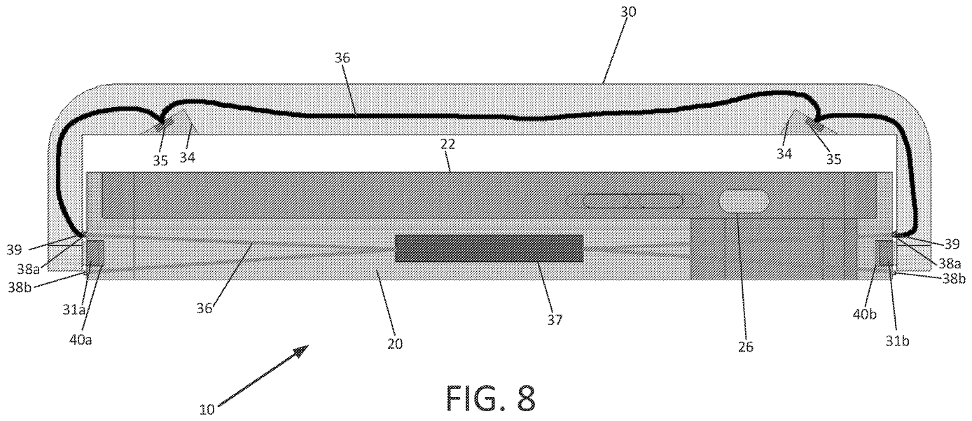

Referring now to the drawings wherein like reference numerals are used to identify identical components in the various views, FIG. 1 is an isometric view of a first configuration of an example sanitizing case 10 for a mobile device. Sanitizing case 10 may include a body 20, an arm 30, a first track 40a, and a second track 40b (shown in FIG. 8). The body 20 may include an interior 21 and walls 22. The bounds of the interior 21 may be defined by the walls 22, which establish a substantially rectangular shape for the interior. The walls 22 include a right wall 22a, left wall 22b, top wall 22c, and bottom wall 22d. The directions of the walls (i.e., left, right) are defined relative to the orientation of the mobile device (not shown) when placed in the interior 21. As shown in FIG. 1, each of the walls 22 may be connected to each other at a curved corner, but other shapes and degrees of corners may be contemplated. To that end, although a particular shape and dimension of the interior 21 is shown, various other shapes and dimensions are contemplated in order to fit and receive a wide range of device shapes and sizes. The body 20 is further shown to include a first aperture 24 and a second aperture 26. In the embodiment shown in FIG. 1, the first aperture 24 is structured to provide an opening for a camera of the mobile device, and the second aperture 26 is structured to provide an opening for a switch of the mobile device. As such, the size, shape, and location of the first aperture 24 and the second aperture 26 may be unique to the mobile device, and other sizes, shapes, and locations of each the first aperture 24 and the second aperture 26 are contemplated in order to adapt the sanitizing case 10 for different mobile devices.

The arm 30 may extend from the top wall 22c to the bottom wall 22d, and a substantially linear portion of the arm 30 may be aligned with the left wall 22b and/or right wall 22a. The arm 30 may include one or more sources of a sanitizing or germicidal agent (e.g., UV light) (shown in FIG. 8) on the substantially linear portion of the arm. The arm 30 may include a first connector 31a that connects to the first track 40a at the bottom wall 22d, and a second connector 31b that connects to the second track 40b at the top wall 22c. The first track 40a may be or may include a slot or similar indentation in the bottom wall 22d that is structured to receive the first connector 31a. As such, the first connector 31a may include a peg, rod, or similar protrusion that is structured to interact with the first track 40a in such a way that the arm 30 is slidably and rotatably coupled to the first track 40a. Similarly, the second connector 31b may include a peg, rod, or similar protrusion that is structured to interact with the second track 40b in such a way that the arm 30 is slidably and rotatably coupled to the second track 40b. As shown in FIG. 1, the arm 30 may be slidably and rotatably coupled to both the first track 40a and the second track 40b. In other embodiments, the arm 30 may be slidably and rotatably coupled to only one of the first track 40a and the second track 40b. Because the arm 30 may be slidably and rotatably coupled to at least one of the first track 40a and the second track 40b, the arm may be able to move orbitally about the entirety of the body 20 by sliding along the first track 40a and/or second track 40b and rotating about the first connector 31a and/or the second connector 31b.

The body 20 may further include a first node 38a and second node 38b, which are raised ridges on either side of the first track 40a that run substantially parallel to and along the length of the first track 40a. An identical set of nodes may be positioned on either side of the second track 40b. Each of the first node 38a and second node 38b may be part of supplying power to the one or more sources of sanitizing or germicidal agent, and are discussed below at greater length with regard to FIG. 8.

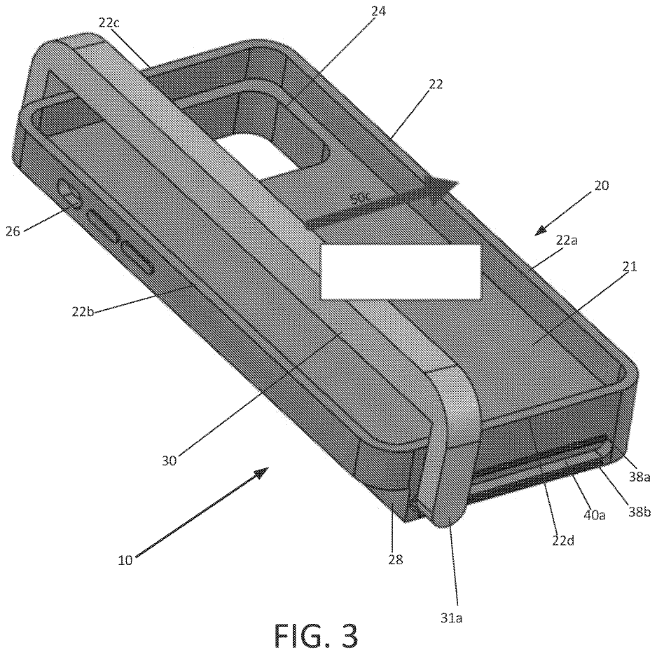

As discussed, the sanitizing case 10 is shown in FIG. 1 in a first configuration. Broadly speaking, the sanitizing case 10 may be structured to sanitize exposed surfaces of the sanitizing case 10 and any device that is secured within the sanitizing case 10 by providing a sanitizing or germicidal agent (e.g., UV light) onto each exposed surface. This is accomplished via movement of the arm 30 orbitally about the sanitizing case 10, with the one or more sanitizing or germicidal agent sources (e.g., LEDs 35) of the arm 30 then providing sanitizing or germicidal agent (e.g., sanitizing UV light) to each exposed surface. As used herein, “configuration” refers generally to the position of the arm 30 relative to the rest of the sanitizing case 10, and “first configuration” refers to the arm 30 being substantially parallel to and flush with the body 20. “Flush” refers to a surface of the arm 30 being in contact with a surface of the body 20, such that there is substantially no space between the arm 30 and the body 20. In some embodiments, such as the one shown in FIG. 1, the sanitizing case 10 includes a cut-out or notch (e.g., slot 28, labelled in FIGS. 3-6) for the arm 30 to slot-in or ‘dock’ with the sanitizing case 10 when in the first configuration. Arrow 50a shows the direction of movement for the arm 30 as the sanitizing case 10 proceeds from the first configuration to a second configuration.

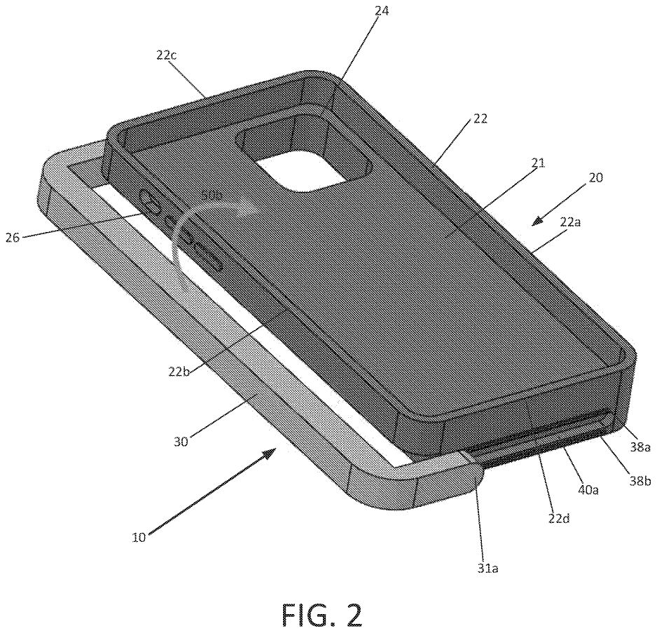

In addition to the various configurations, the arm 30 of the sanitizing case 10 may have a first position and a second position. In the first position, which is analogous to the first configuration, the arm 30 is secured to the sanitizing case 10 (e.g., stowed within the slot 28) and movement about the sanitizing case 10 is restricted. In the second position, which is analogous to the remaining configurations, the arm 30 is disposed or moved away from the sanitizing case 10 and movement about the sanitizing case 10 is restricted only by the bounds of the first track 40a and/or the second track 40b (i.e., the arm 30 is able to move without restriction along the tracks). Put differently, when the sanitizing case 10 is in the first configuration, the arm 30 is in the first position, and when the sanitizing case 10 is in any of the second through seventh configurations (illustrated in FIGS. 2-7), the arm 30 is in the second position. In these embodiments, the sanitizing case 10 may include a locking mechanism configured to secure the arm 30 in the first position. In some embodiments, the arm 30 may be biased to the second position (e.g., spring-loaded), so when the locking mechanism is disengaged, the arm 30 moves automatically to the second position. In other embodiments, the arm 30 is not biased to either position, so disengaging the locking mechanism allows the arm 30 to be moved to the second position, but such movement does not happen automatically.

FIG. 2 is an isometric view of the second configuration of the sanitizing case 10. As discussed above with reference to FIG. 1, the sanitizing case 10 adjusts from the first configuration to the second configuration by moving the arm 30 according to the direction indicated by arrow 50a of FIG. 1. Therefore, the second configuration here is shown to be when the arm 30 is displaced from the rest of the sanitizing case 10 but still substantially parallel with the length of the first track 40a (i.e., the arm 30 slid along the first track 40a and/or second track 40b without rotation). Arrow 50b shows the direction of movement for the arm 30 as the sanitizing case 10 proceeds from the second configuration to a third configuration.

FIG. 3 is an isometric view of the third configuration of the sanitizing case 10. As discussed above with reference to FIG. 2, the sanitizing case 10 adjusts from the second configuration to the third configuration by moving the arm 30 according to the direction indicated by arrow 50b of FIG. 2. Therefore, the third configuration here is shown to be when the arm 30 is substantially perpendicular to the axis formed by the length of the first track 40a (i.e., the arm 30 rotated 90 degrees about the first connector 31a). When a mobile device is secured within the interior 21 and when the sanitizing case 10 is in the third configuration, the one or more sanitizing agent sources of the arm 30 are providing sanitizing agent onto a surface (e.g., screen) of the mobile device. Arrow 50c shows the direction of movement for the arm 30 as the sanitizing case 10 proceeds from the third configuration to a fourth configuration.

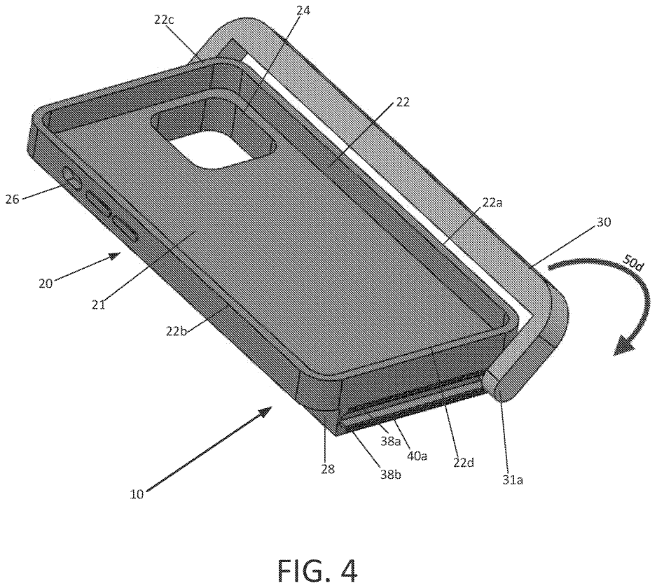

FIG. 4 is an isometric view of the fourth configuration of the sanitizing case 10, in accordance with various embodiments of the present invention. As discussed above with reference to FIG. 3, the sanitizing case 10 adjusts from the third configuration to the fourth configuration by moving the arm 30 according to the direction indicated by arrow 50c of FIG. 3. Therefore, the fourth configuration here is shown to be when the arm 30 is still displaced from the rest of the sanitizing case 10 and in the middle of a rotation about the right wall 22a (i.e., the arm 30 slid along the entire length of the first track 40a and/or second track 40b and started rotating). Arrow 50d shows the direction of movement for the arm 30 as the sanitizing case 10 proceeds from the fourth configuration to a fifth configuration.

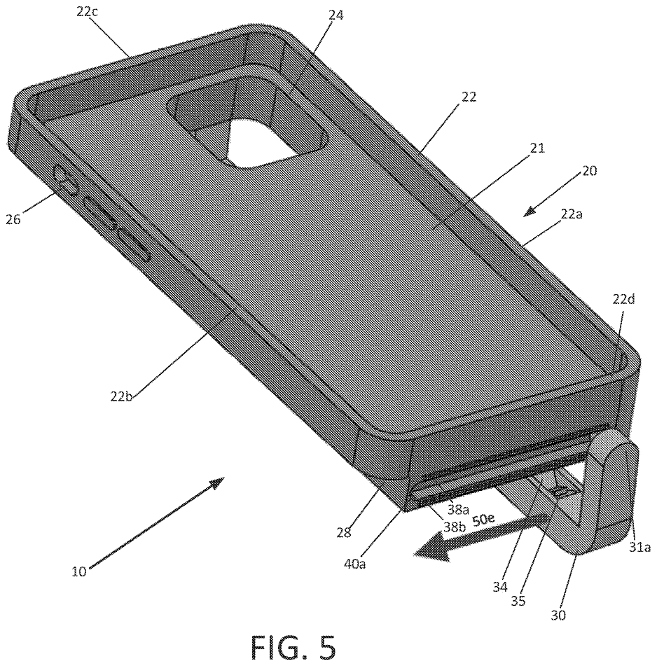

FIG. 5 is an isometric view of the fifth configuration of the sanitizing case 10, in accordance with various embodiments of the present invention. As discussed above with reference to FIG. 4, the sanitizing case 10 adjusts from the fourth configuration to the fifth configuration by moving the arm 30 according to the direction indicated by arrow 50d of FIG. 4. Therefore, the fifth configuration here is shown to be when the arm 30 is substantially perpendicular to the axis formed by the first track 40a, similar to the third configuration. However, in contrast to the third configuration, the one or more sanitizing agent sources of the arm 30 in the fifth configuration are providing sanitizing agent to the back of the sanitizing case (i.e., the part of the sanitizing case 10 that covers the back of the mobile phone). Arrow 50e shows the direction of movement for the arm 30 as the sanitizing case 10 proceeds from the fifth configuration to a sixth configuration.

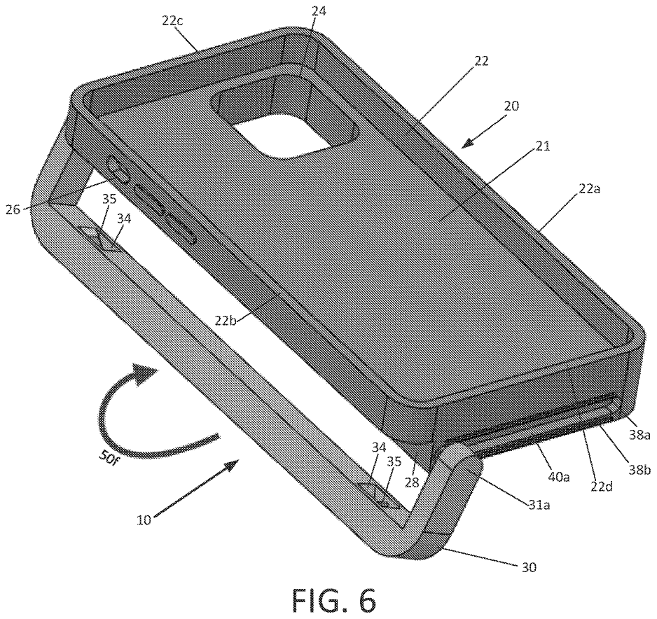

FIG. 6 is an isometric view of the sixth configuration of the sanitizing case 10, in accordance with various embodiments of the present invention. As discussed above with reference to FIG. 5, the sanitizing case 10 adjusts from the fifth configuration to the sixth configuration by moving the arm 30 according to the direction indicated by arrow 50e of FIG. 5. Therefore, the sixth configuration here is shown to be when the arm 30 is displaced from the rest of the sanitizing case 10 and in the middle of a rotation about the left wall 22b (i.e., the arm 30 slid back along the entire length of the first track 40a and/or second track 40b and started rotating). Arrow 50f shows the direction of movement for the arm 30 as the sanitizing case 10 proceeds from the sixth configuration to a seventh configuration.

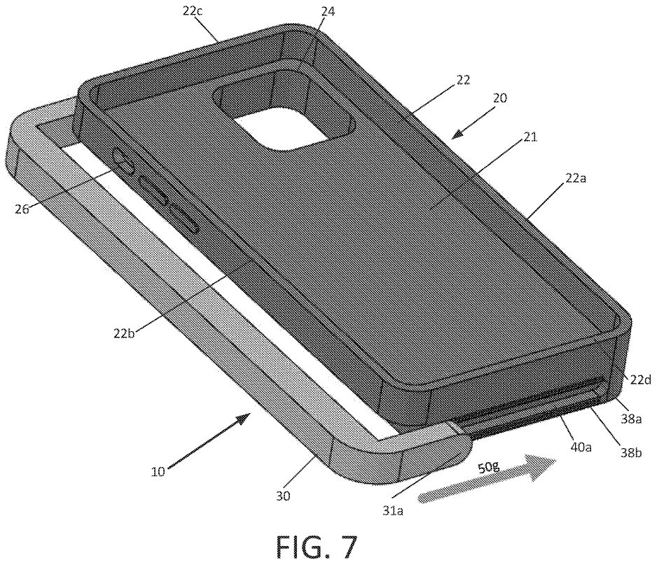

FIG. 7 is an isometric view of the seventh configuration of the sanitizing case 10, in accordance with various embodiments of the present invention. As discussed above with reference to FIG. 6, the sanitizing case 10 adjusts from the sixth configuration to the seventh configuration by moving the arm 30 according to the direction indicated by arrow 50f of FIG. 6. Therefore, the seventh configuration here is shown to be when the arm 30 is displaced from the rest of the sanitizing case 10 and substantially parallel with an axis formed by the length of the first track 40a. As shown, the seventh configuration is statically identical to the second configuration—the only difference in the two configurations is the progression of configurations that lead into each (i.e., the order of the configuration within the sanitizing process). The sanitizing case 10 adjusts into the second configuration by moving the arm 30 from the docked position of the first configuration, while the sanitizing case 10 adjusts into the seventh configuration by completing a rotation started in the sixth configuration. As such, even though the second and seventh configurations appear identical, they differ in the configurations that come immediately before. Arrow 50g shows the direction of movement for the arm 30 as the sanitizing case 10 proceeds from the seventh configuration to the first configuration.

In the sanitizing case 10, the one or more sources of sanitizing or germicidal agent may be two ultraviolet (UV) LEDs 35. LEDs 35 may operate at the germicidal range of the ultraviolet light spectrum and/or have a germicidal coating. In embodiments where LEDs 35 apply germicidal UV as a sanitizing agent, LEDs 35 may operate within a wide range of powers and exposure times, such as, for example, power in the range of about 4-65 watts and exposure times of about 2-10 seconds. However, it should be understood that less power may be used in conjunction with a longer exposure time, or more power in conjunction with a shorter exposure time. LEDs 35 are not limited to germicidal UV as a sanitizing agent. LEDs 35 may sanitize surfaces based on one or more of: (1) a particular characteristic of the agent or energy applied (e.g., germicidal UV); (2) the heat resulting from the application of the sanitizing agent or energy, or (3) the interaction of the agent with a particular characteristic of the surface or another substance applied to the surface.

As shown in FIG. 5, the LED 35 may be mounted within a recess 34. The recess(es) 34 may feature angled sidewalls that define the edges of propagation of the sanitizing agent (e.g., germicidal UV light) and control the dispersion of light rays from the LEDs 35. In this manner, the recesses 34 direct the light from the LEDs 35 in a desired direction and with a desired area of effect, which can improve the effectiveness of the LEDs 35 and of the case 10 in general. Furthermore, the angled sidewalls of the recesses 34 prevent light from the LEDs 35 from dispersing away from the mobile device, which reduces the possibility that users (or bystanders) are exposed to light. Because germicidal UV light can be harmful to eyes or skin, the recesses 34 may be included as a safety feature to reduce the risk of harmful exposure.

FIG. 8 is a schematic view of the third configuration for the sanitizing case 10, in accordance with various embodiments of the present invention. As shown in FIG. 8, the body 20 includes a power supply 37. In some embodiments, the power supply 37 may be one or more battery packs (e.g., 6-volt battery packs) included within the body 20. In other embodiments, the power supply 37 may be a power supply of the secured mobile device, such that the power supply of the secured mobile device provides power to the sanitizing case 10 (e.g., via a connecting cable, port, etc.). The power supply 37 is connected to the LEDs 35 via one or more cables 36.

In some embodiments, such as the embodiment shown in FIG. 8, the power supply 37 is connected to the LEDs 35 only when the first node 38a or the second node 38b are in contact with an arm node 39 of the arm 30. Put differently, the ‘circuit’ formed by the power supply 37, cables 36, and LEDs 35 is completed only when the arm node 39 is in contact with the first node 38a or second node 38b. Each of the first node 38a, second node 38b, and arm node 39 may be protrusions of conductive material that are structured to promote and facilitate the provision of electric current. As shown in FIGS. 1-7, the first node 38a and second node 38b are raised ridges that run along the length of the first track 40a and second track 40b. The arm node 39 is shown as being located on the LED-side (i.e., the side most proximate to the linear portion of the arm 30 on which the LEDs 35 are located) of the first connector 31a and second connector 31b. In this particular embodiment then, the arm node 39 is in contact with the first node 38a only when the arm 30 is substantially perpendicular to the screen of the any secured mobile device (i.e., the sanitizing case 10 is in the third configuration of FIG. 3), and is in contact with the second node 38b only when the arm is substantially perpendicular to the back of the sanitizing case (i.e., the sanitizing case 10 is in the fifth configuration of FIG. 5). Put differently, the sanitizing case 10 begins sanitizing in the second configuration and continues until the third configuration, and then resumes sanitizing in the fifth configuration and ceases sanitizing at the sixth configuration. In other embodiments, the LEDs 35 may be selectively engaged and disengaged with a button or switch on the case 10 or arm 30, while in further embodiments, the LEDs 35 may be automatically engaged for a period of time when the arm 30 is undocked (i.e., moved from the first configuration to the second configuration).

As such, the sanitizing case 10 may have at least two operating states: a first state in which the LEDs 35 directs an amount of a sanitizing agent towards the sanitizing case 10 and/or secured mobile device, and a second state in which the LEDs 35 do not direct any sanitizing agent toward the same location. The operating state may be determined based on the presence of contact between the first node 38a or the second node 38b with the arm node 39, such that the sanitizing case 10 is in the first state when the nodes are in contact and in the second state when there is no contact. In some embodiments, a switch is included in order to manually change the operating state of the sanitizing case 10. In these embodiments, the switch is configured to selectively complete or break the circuit formed by the power supply 37, cables 36, and LEDs. The manual switch may be integrated into the sanitizing case 10 (such as in body 20). The switch may be a button, lever, switch, other binary switching device, or a slider, dimmer, or other analog switching device

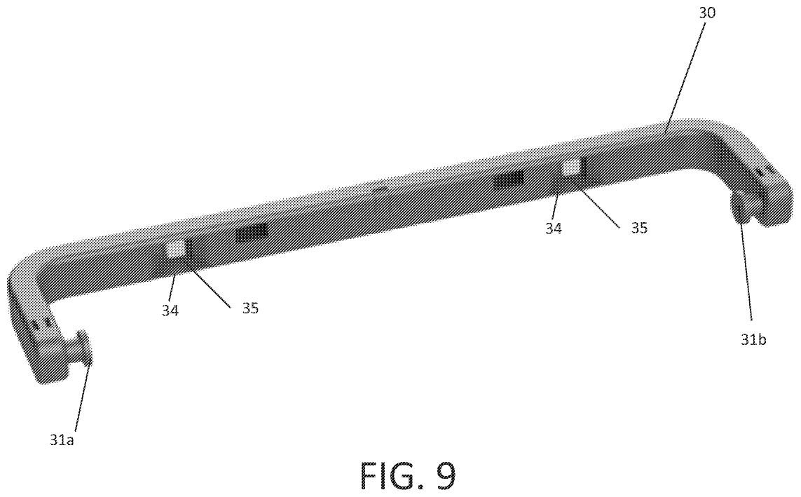

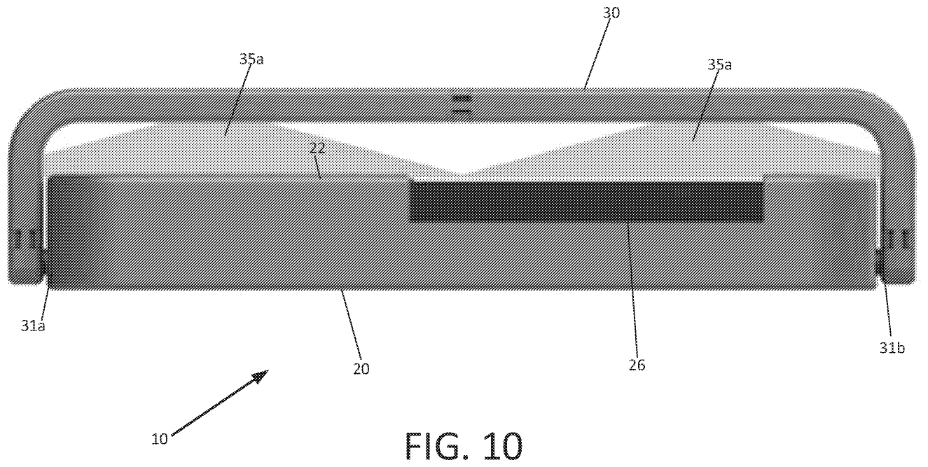

FIG. 9 is a schematic view of the sanitizing case 10 showing the dispersal of the sanitizing agent from the LEDs 35, in accordance with various embodiments of the present invention. Each LED 35 may generate a light dispersal field 35a, and the light dispersal fields may collectively cover the entire length of the mobile device and the case 10, in some embodiments.

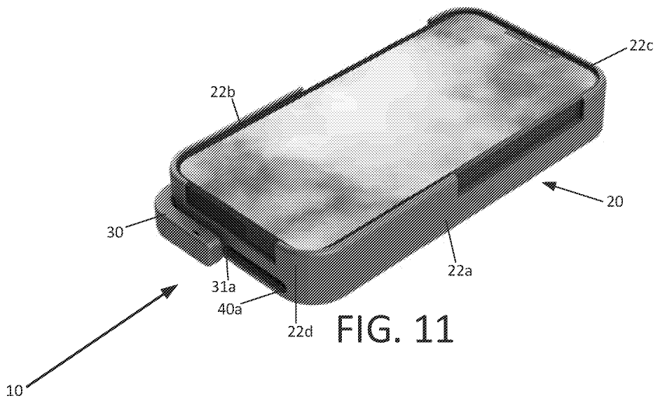

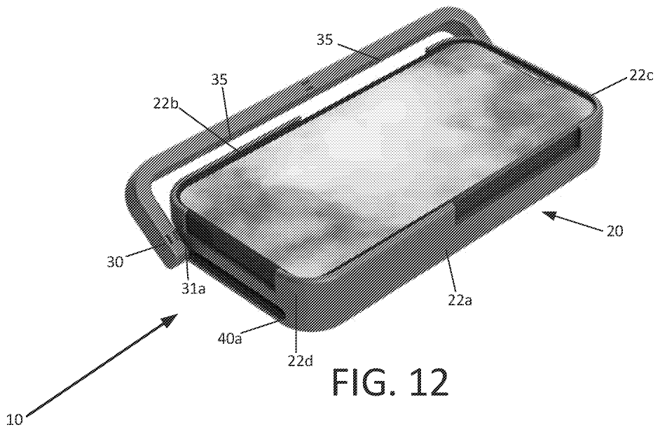

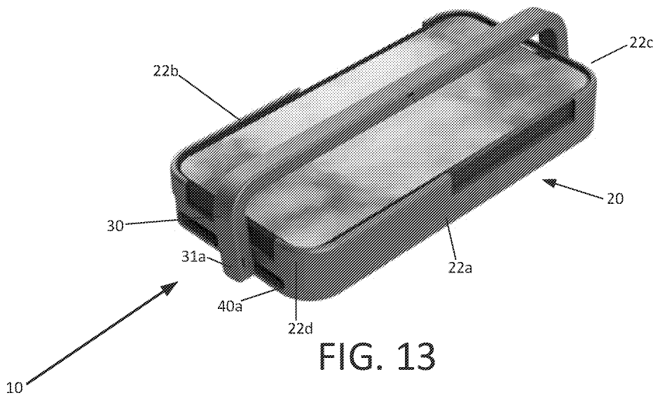

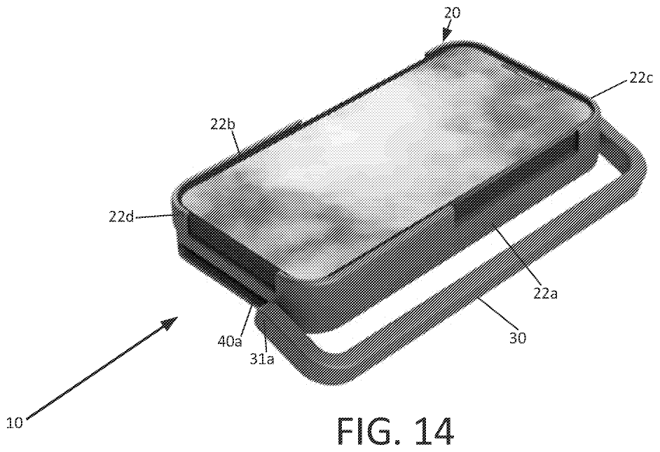

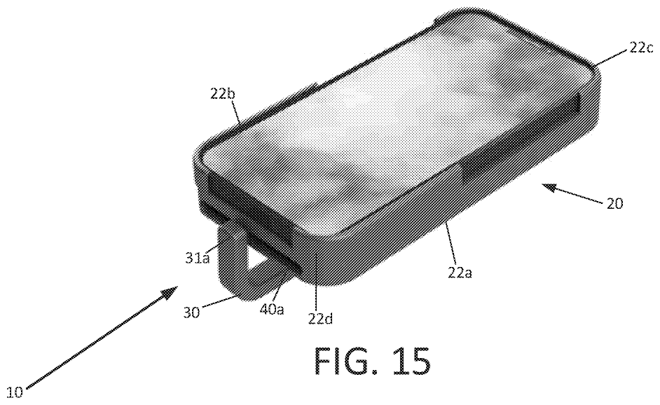

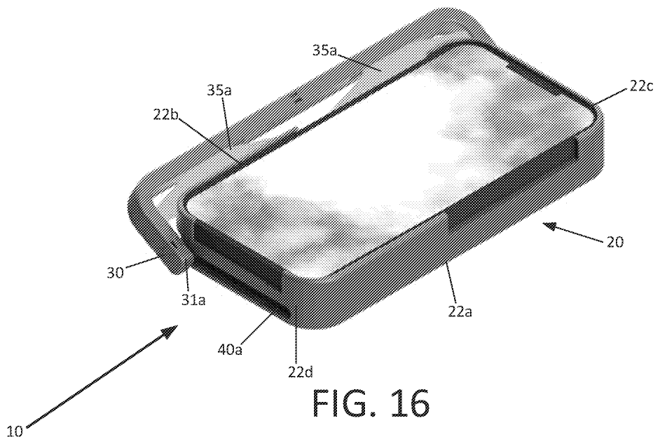

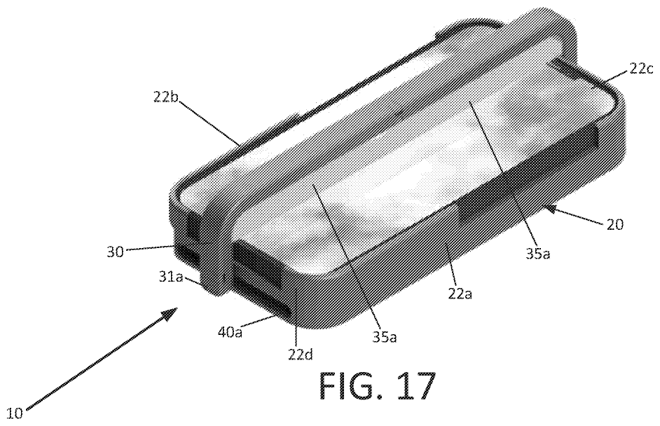

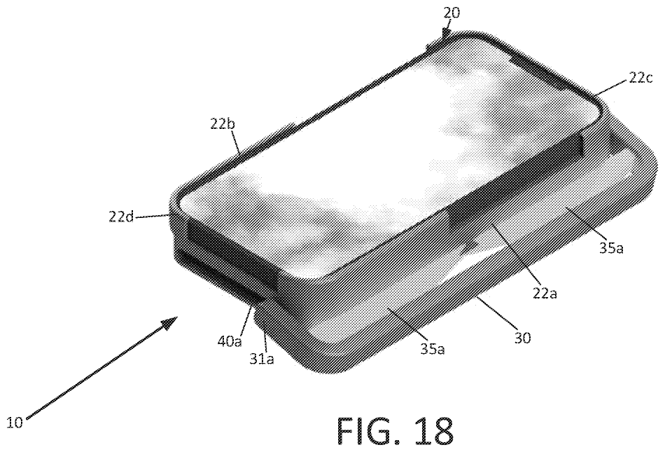

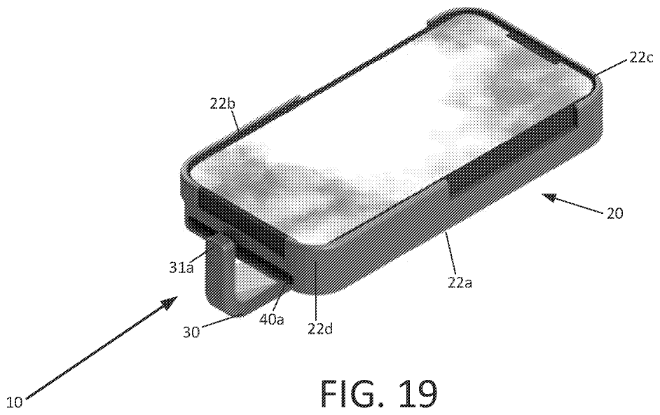

FIGS. 11-19 are additional views of the case 10, further illustrating the features and functionality described herein.

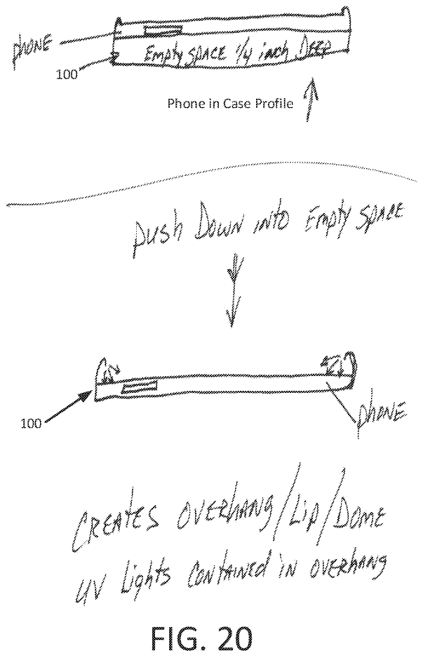

FIG. 20 includes side views of an example sanitizing case. As shown in FIG. 20, when a mobile device (e.g., a phone) is in a first position in the case 100, there is an empty space between a back of the phone and a rear or bottom wall of the case 100. The phone may be held in this first position by one or more ridges or protrusions within the case 100. In some embodiments, the phone may be placed in a slider(s) or similar component that is slidably coupled to the interior of the case 100, such that when the phone is coupled to the slider(s), the phone is able to slide within the case 100 (e.g., from the first position to a second position). As further shown in FIG. 20, when the phone is in the second position, the phone is now flush with the back wall of the case 100, and the empty space is now between the face of the phone and a lip of the case 100. The lip of the case 100 may include one or more sanitizing or germicidal agent sources (e.g., UV light bulbs) that are configured to apply sanitizing or germicidal agent (e.g., UV light) to the screen of the phone. As such, when the phone is in the first position, the screen of the phone is elevated for use by the owner, and when the phone is in the second position, the screen of the phone may be sanitized by the UV lights.

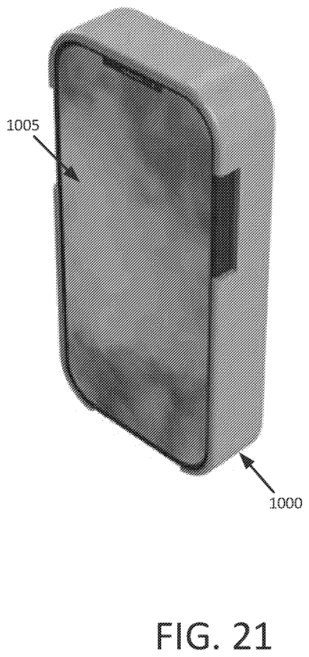

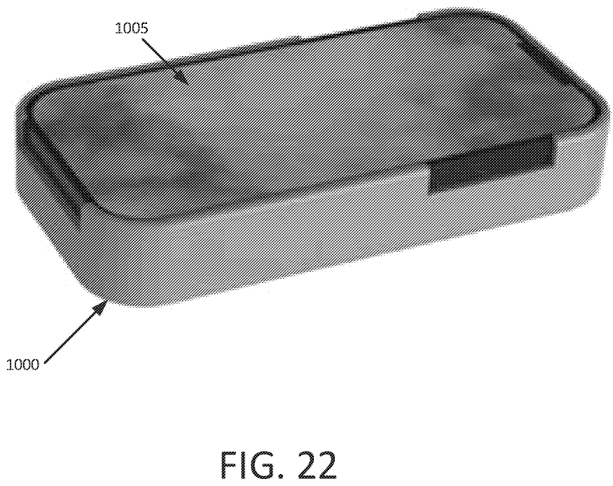

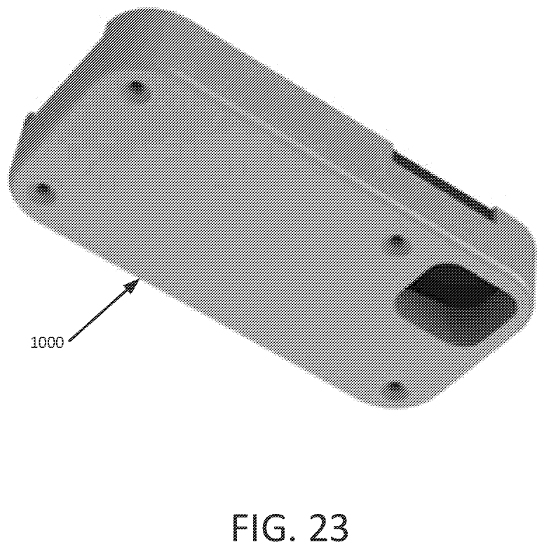

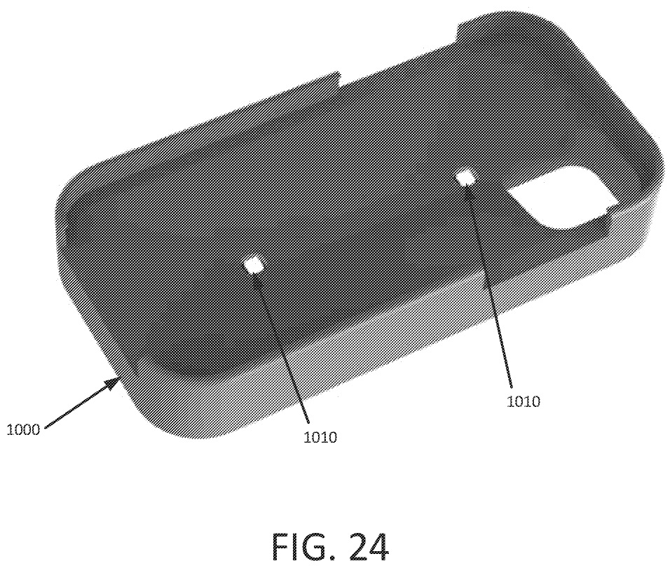

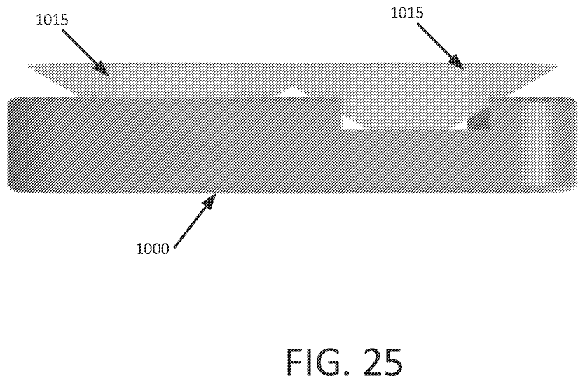

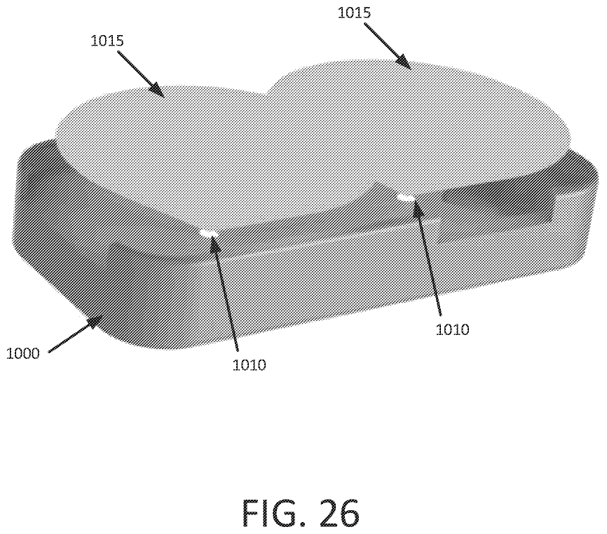

FIGS. 21-26 are views of an example sanitizing case 1000. As shown in FIGS. 21 and 22, when a mobile device 1005 is in the case 1000, a face or screen of the mobile device 1005 is flush with the case 1000, such that access to the entire screen is unobstructed while the mobile device 1005 is in the case 1000. The case 1000 is structured to receive the mobile device 1005 in a first orientation in which the screen of the mobile device 1005 is visible and usable (e.g., the orientation shown in FIGS. 21 and 22) and in a second orientation in which a back of the mobile device 1005 is visible and the screen is not. Referring now to FIGS. 24-26, which show the case 1000 without the mobile device 1005, the case 1000 includes one or more LEDs 1010 (analogous to which may be the same as LEDs 35, in some embodiments) that each may generate light dispersal fields 1015. The light dispersal fields 1015 may collectively cover the entire length of the mobile device 1005, such that the entire front or back of the mobile device 1005 may be sanitized by the LEDs 1010. Furthermore, the case 1000 may be configured to engage the one or more LEDs 1010 only if the mobile device 1005 is in place, such that the LEDs 1010 are not engaged when there is a possibility that the LEDs 1010 could expose a user or bystander to the light dispersal fields 1015. Because the light from the LEDs 1010 could cause damage to eyes, the case 1000 may include such a safety feature in order to engage the LEDs 1010 only when unintentional exposure to the light dispersal fields 1015 is minimized or eliminated.

A system and apparatus for sanitizing a mobile electronic device, such as, for example, a cell phone, tablet, portable music device, portable gaming system, or another mobile device, as described herein has many advantages. Such mobile devices carry many communicable germs, as they are often used without thought and in all circumstances. By reducing the amount of communicable germs that a mobile device is carrying, users may be better protected and can feel more comfortable using their mobile device, even after exposure to germ-heavy environments, such as public transit or a crowded school. Integrating a sanitizing device into a mobile device case enables a user to have access to sanitizing agent at all times, which enables a user to sanitize their phone throughout the day rather than at a single time or place. For example, a user may sanitize their phone as soon as they disembark from a public train rather than having to wait to sanitize until they have returned home from a work-day. Furthermore, the integration of the sanitizing agent source into a device case reduces costs and inconvenience for a user by reducing the number of overall devices the user must purchase and keep track of—a single device is able to protect their mobile phone from both bumps and germs.

Although numerous embodiments of this invention have been described above with a certain degree of particularity, those skilled in the art could make numerous alterations to the disclosed embodiments without departing from the spirit or scope of this invention. All directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, configuration, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A mobile electronic device case, the case comprising:
a body defining an interior, the interior configured to secure a mobile electronic device;
an arm movably coupled to the body; and
a germicidal agent source disposed on the arm,
wherein the arm is movable relative to the mobile electronic device for applying the germicidal agent to the mobile electronic device when the mobile electronic device is secured in the body.

2. The case of claim 1, wherein the arm is at least one of slidably or rotatably coupled to the body.

3. The case of claim 1, wherein:
the body defines a track, and
the arm slides along the track.

4. The case of claim 3, wherein the arm is rotatably coupled to the track.

5. The case of claim 3, wherein the track comprises:
a first track portion disposed along a first side of the body, and
a second track portion disposed along a second side of the body;
wherein the arm slides along the first track portion and the second track portion.

6. The case of claim 3, wherein:
the arm comprises a first position and a second position,
in the first position, the arm is substantially adjacent to the body and movement of the arm along and about the track is limited, and
in the second position, the arm is disposed away from the body and movement of the arm along and about the track is unrestricted.

7. The case of claim 6, wherein the arm is secured in the first position via a spring-loaded mechanism.

8. The case of claim 3, wherein the arm is configured to move orbitally about the case via rotation about and sliding along the track,
whereby the germicidal agent source provides the germicidal agent on each exposed surface of the case and of the mobile electronic device.

9. The case of claim 1, wherein the arm comprises a linear portion configured to be substantially parallel to a screen of the mobile electronic device when the mobile electronic device is secured in the body.

10. The case of claim 1, wherein the arm extends from a top of the case to a bottom of the case and is substantially parallel to a lateral side of the case.

11. The case of claim 1, wherein the germicidal agent source is disposed on the arm within a recess, the recess featuring one or more angled sidewalls that reduce a dispersion area of the germicidal agent.

12. The case of claim 1, further comprising a connector configured to electronically couple the case with a power supply of the mobile electronic device.

13. A mobile electronic device case, the case comprising:
a body defining an interior and a track, the interior configured to secure a mobile electronic device;
an arm movably coupled to the body; and
a germicidal agent source disposed on the arm,
wherein the arm is slidably and rotatably coupled to the track, and movable relative to the mobile electronic device for applying the germicidal agent to each exposed surface of the case and of the mobile electronic device when the mobile electronic device is secured in the body.

14. The case of claim 13, wherein the track comprises:
a first track portion disposed along a first side of the body, and
a second track portion disposed along a second side of the body;
wherein the arm slides along the first track portion and the second track portion.

15. The case of claim 13, wherein:
the arm comprises a first position and a second position,
in the first position, the arm is substantially adjacent to the body and movement of the arm along and about the track is limited, and
in the second position, the arm is disposed away from the body and movement of the arm along and about the track is unrestricted.

16. The case of claim 13, wherein the arm further comprises a linear portion configured to be substantially parallel to a screen of the mobile electronic device when the mobile electronic device is secured in the body.

17. The case of claim 13, further comprising a power supply contained within the case and configured to be selectively coupled to the germicidal agent source.

18. The case of claim 13, further comprising a connector configured to electronically couple the case with a power supply of the mobile electronic device.

19. A method for sanitizing a mobile electronic device, comprising:
providing a case for the mobile electronic device, the case comprising:
a body, the body defining a track and an interior configured to secure the mobile electronic device;
an arm movably coupled to the track; and
a germicidal agent source disposed on the arm;
activating the germicidal agent source; and
moving the arm along the track to sanitize one or more surfaces of the case and of the mobile electronic device.

20. The method of claim 19, wherein:
the arm comprises a first position and a second position,
in the first position, the arm is substantially adjacent to the body and movement of the arm along and about the track is limited,
in the second position, the arm is disposed away from the body and movement of the arm along and about the track is unrestricted, and
the method further comprises, prior to moving the arm along the track:
disengaging a locking mechanism securing the arm in the first position; and
moving the arm from the first position to the second position.
